SEQUENCE LISTING (2 sequences)

SEQ ID NO: 1 - protein, 664 aa, Mycobacterium tuberculosis
MSDAVPQVGSQFGPYQLLRLLGRGGMGEVYEAEDTRKHRVVALKLISPQYSDNAVFRARMQREADTAGRLTEPHIVPIHDYGEINGQFFVEMRMIDGTSLRALLKQYGPLTPARAVAIVRQIAAALDAAHANGVTHRDVKPENILVTASDFAYLVDFGIARAASDPGLTQTGTAVGTYNYMAPERFTGDEVTYRADIYALACVLGECLTGAPPYRADSVERLIAAHLMDPAPQPSQLRPGRVPPALDQVIAKGMAKNPAERFMSAGDLAIAAHDALTTSEQHQATTILRRGDNATLLATPADTGLSQSESGIAGAGTGPPTPGAARWSPGDSATVAGPLAADSRGGNWPSQTGHSPAVPNALQASLGHAVPPAGNKRKVWAVVGAAAIVLVAIVAAAGYLVLRPSWSPTQASGQTVLPFTGIDFRLSPSGVAVDSAGNVYVTSEGMYGRVVKLATGSTGTTVLPFNGLYQPQGLAVDGAGTVYVTDFNNRVVTLAAGSNNQTVLPFDGLNYPEGLAVDTQGAVYVADRGNNRVVKLAAGSKTQTVLPFTGLNDPDGVAVDNSGNVYVTDTDNNRVVKLEAESNNQVVLPFTDITAPWGIAVDEAGTVYVTEHNTNQVVKLLAGSTTSTVLPFTGLNTPLAVAVDSDRTVYVADRGNDRVVKLTS

SEQ ID NO: 2 - protein, 262 aa, Mycobacterium tuberculosis
RPSWSPTQASGQTVLPFTGIDFRLSPSGVAVDSAGNVYVTSEGMYGRVVKLATGSTGTTVLPFNGLYQPQGLAVDGAGTVYVTDFNNRVVTLAAGSNNQTVLPFDGLNYPEGLAVDTQGAVYVADRGNNRVVKLAAGSKTQTVLPFTGLNDPDGVAVDNSGNVYVTDTDNNRVVKLEAESNNQVVLPFTDITAPWGIAVDEAGTVYVTEHNTNQVVKLLAGSTTSTVLPFTGLNTPLAVAVDSDRTVYVADRGNDRVVKLTS

(19) United States
(12) Patent Application Publication
(10) Pub. No.: US 2016/0136255 A1
(43) Pub. Date: May 19, 2016

(54) VACCINATION WITH RECOMBINANT MYCOBACTERIUM TUBERCULOSIS PknD ATTENUATES BACTERIAL DISSEMINATION TO THE BRAIN

(71) Applicant: THE JOHNS HOPKINS UNIVERSITY, Baltimore, MD (US)

(72) Inventors: SANJAY K. JAIN, BALTIMORE, MD (US); CIARAN SKERRY, BALTIMORE, MD (US); NICHOLAS A. BE, OAKLAND, CA (US)

(73) Assignee: THE JOHNS HOPKINS UNIVERSITY, BALTIMORE, MD (US)

(21) Appl. No.: 14/897,736

(22) PCT Filed: Jun. 6, 2014
(86) PCT No.: PCT/US14/41327

§ 371 Date: Dec. 11, 2015

Related U.S. Application Data
(60) Provisional application No. 61/833,606, filed on Jun. 11, 2013.

(51) Int. Cl. A61K 39/04

(52) U.S. Cl. CPC A61K 39/04; A61K 2039/55511

(57) ABSTRACT

Vaccines comprising Mycobacterium tuberculosis (M. tuberculosis) PknD sensor polypeptides, compositions, kits, and methods of use for treating central nervous system tuberculosis are disclosed. More specifically, the disclosure provides a method for treating or preventing central nervous system (CNS) tuberculosis (TB) in a subject, the method comprises administering to the subject a therapeutically effective amount of a vaccine comprising an M tuberculosis PknD sensor polypeptide, or an immunogenic fragment for inhibiting or preventing M tuberculosis bacterial invasion of brain microvascular endothelia.

### CROSS-REFERENCE TO RELATED APPLICATIONS

This application claims the benefit of U.S. Provisional Application No. 61/833,606, filed Jun. 11, 2013, which is incorporated herein by reference in its entirety.

### FEDERALLY SPONSORED RESEARCH OR DEVELOPMENT

This invention was made with government support under OD006492 and AI083125 awarded by the National Institutes of Health. The government has certain rights in the invention.

### BACKGROUND

Central nervous system (CNS) tuberculosis (TB) is a serious, often fatal disease that predominantly affects young children (Lincoln et al., 2003; Jacobs and Starke, 2003; Girgis et al., 1998). Two major forms of CNS TB include meningitis, which accounts for 0.5% to 1% of all TB disease, and intra-cranial tuberculomas, which on a global level account for up to 40% of ‘brain tumors’ (Jacobs et al., 2003; Jain et al., 2005). Co-infection with HIV not only increases the risk of development of CNS TB (Berenguer et al., 1992; Rana et al., 2000), but also leads to a much higher case-fatality rate (Katrak et al., 2000; Thwaites et al., 2004). Non-specific clinical presentation, poor diagnostics, and delays in instituting appropriate TB treatment (for example, drug susceptibility tests take up to 10 weeks) complicate CNS TB leading to severe, irreversible neurological damage and high mortality. Treatment of CNS TB becomes even more challenging in the age of multi-drug (MDR), extensively-drug (XDR), and totally-drug resistant (TDR) strains of Mycobacterium tuberculosis (Udwadia, 2012). Moreover, several second line TB drugs have limited CNS penetration. Since clinical outcomes (even with appropriate treatment) after the onset of TB meningitis are extremely poor (Thwaites, 2005; Sofia et al., 2001; Padayatchi et al., 2006), developing preventive strategies against CNS TB is a high priority.

BCG is the only licensed vaccine against tuberculosis (TB) and is recommended for administration to all newborns at birth by the World Health Organization (WHO), in countries with a high TB burden (WHO, 1993). The protection offered by BCG for adult pulmonary TB, however, is highly variable (0-80%) (Rosenthal et al., Am Rev Respir Dis, 1961; Rosenthal et al., Pediatrics, 1961), and the current WHO recommendation is based on the ability of BCG to protect against severe forms of TB, such as TB meningitis, during infancy (WHO, 1993). BCG works by the induction of T-cell immune responses that limit M. tuberculosis burden at the site of primary infection (mostly lungs). Since the magnitude of the pulmonary bacterial burden determines extrapulmonary dissemination, the reduction in the pulmonary burden due to BCG, decreases extrapulmonary hematogenous dissemination of bacteria and subsequent risk of developing CNS TB and meningitis (WHO, 1993). Unfortunately, protection offered by BCG against TB meningitis also is quite variable (52-100%) (Romanus, 987; Padungchan et al., 1986; Tidjani et al., 1986; Young and Hershfield, 1986). BCG is not well-defined antigenically, and the several different BCG strains in clinical use, offer variable levels of protection (Ritz et al., 2012).

Moreover, BCG is a live vaccine, and therefore may be unsuitable for immunosuppressed infants especially in the setting of HIV (Ottenhoff and Kaufmann, 2012). BCG vaccination also confounds the interpretation of the tuberculin skin test, and is an additional limitation, which would not apply to subunit vaccines. For these reasons, a new, preferably acellular, alternative to BCG vaccination would be ideal. To this end, several acellular candidates exist (Parra et al., 2004; Campuzano et al., 2007; Wang et al., 2007), though none have explored the ability to prevent CNS TB.

### SUMMARY

In some aspects, the presently disclosed subject matter provides a vaccine comprising an M. tuberculosis PknD sensor polypeptide, or an immunogenic fragment thereof, wherein the M. tuberculosis PknD sensor polypeptide or immunogenic fragment thereof inhibits or prevents M. tuberculosis bacterial invasion of brain microvascular endothelia. In certain aspects, the M. tuberculosis PknD sensor polypeptide comprises an amino acid sequence that is at least 70% identical, e.g., at least 75%, 80%, 85%, 90%, 91%, 92%, 93%, 94%, 95%, 96%, 97%, 98%, or 99% identical to SEQ ID NO:2. In another aspect, the PknD sensor polypeptide, or an immunogenic fragment thereof, is recombinant. In another aspect, the vaccine further comprises an adjuvant, particularly dimethyl dioctadecyl-ammonium bromide (DDA). In another aspect, the vaccine further comprises a physiologically compatible carrier. In yet another aspect, the vaccine further comprises BCG.

In other aspects, the presently disclosed subject matter provides a method of treating or preventing central nervous system (CNS) tuberculosis (TB) in a subject in need thereof, the method comprising administering to the subject a therapeutically effective amount of a vaccine comprising an M. tuberculosis PknD sensor polypeptide, or an immunogenic fragment thereof, wherein the M. tuberculosis PknD sensor polypeptide or immunogenic fragment thereof inhibits or prevents M. tuberculosis bacterial invasion of brain microvascular endothelia. In certain aspects, the M. tuberculosis PknD sensor polypeptide for treating or preventing CNS TB in a subject in need thereof comprises an amino acid sequence that is at least 70% identical, e.g., at least 75%, 80%, 85%, 90%, 91%, 92%, 93%, 94%, 95%, 96%, 97%, 98%, or 99% identical to SEQ ID NO:2. In other aspects, the method for treating or preventing CNS TB in a subject in need thereof further comprises administering an additional vaccine to the subject, particularly wherein the additional vaccine is BCG. In additional aspects, administering the vaccine comprising an M. tuberculosis PknD sensor polypeptide, or an immunogenic fragment thereof, is either prior to administering BCG, subsequent to administering BCG, or simultaneous with administering BCG. In some aspects the CNS TB is meningitis, while in other aspects the CNS TB is intracranial tuberculoma.

In other aspects, the presently disclosed subject matter provides a kit comprising a vaccine comprising an M. tuberculosis PknD sensor polypeptide, or an immunogenic fragment thereof, wherein the kit further comprises a set of instructions for administering the vaccine in a therapeutically effective amount for treating or preventing CNS TB in a subject in need thereof. In another aspect, the kit further comprises an additional vaccine, particularly BCG, wherein the set of instructions further include methods for concurrent administration.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIGS. 1A and 1B demonstrate that PknD vaccination protects against M. tuberculosis dissemination to the brain. Colony-forming units (CFU) in the lungs (panel A) and brains (panel B) of guinea pigs vaccinated with BCG (triangle), recombinant PknD (inverted triangle), DDA (adjuvant alone) (square) or PBS (unvaccinated controls) (circle), 4- and 6-weeks after an aerosol challenge with M. tuberculosis are shown. BCG vaccination limited the pulmonary bacillary load (P=0.01) and also significantly reduced the bacillary burden in the brains after aerosol challenge with virulent M. tuberculosis (P=0.01). While PknD vaccination did not limit bacillary growth in the lungs, it offered significant protection against bacillary dissemination to the brain, which was no different from BCG (P>0.24), even though the PknD vaccinated animals had almost 100-fold higher bacterial burdens in the lungs. Four guinea pigs per group were sacrificed at each time-point, except for the DDA group, for which only three animals were sacrificed 6-weeks after aerosol challenge. Data are presented on a logarithmic scale as mean±standard deviation;

FIGS. 2A and 2B demonstrate that PknD vaccination induces specific IFN-γ and IgG responses. Splenic recall assays were performed on splenocytes from guinea pigs vaccinated with BCG, recombinant PknD, DDA (adjuvant alone) or PBS (unvaccinated controls). The WST-1 system was then used to measure proliferation after stimulation heat-inactivated M. tuberculosis (white bars), recombinant PknD subunit (striped bars) or control with culture media alone (black bars) (panel A). Significant cellular proliferation in response to PknD subunit was only observed in splenocytes from PknD vaccinated animals (P=0.002). Similarly, significant proliferation in response to heat-inactivated M. tuberculosis was only observed in splenocytes from BCG vaccinated animals (P=0.01). Supernatants from these assays were also used to measure the IFN-γ levels (panel B). Consistent with proliferation data, only animals vaccinated with PknD (P<0.01) generated appreciable levels of IFN-γ in response to the PknD subunit protein (FIG. 2B). Splenocytes from three animals from each group were tested. Each assay was performed in triplicate. Data are presented on a linear scale as mean±standard deviation;

FIGS. 3A and 3B show M. tuberculosis PknD-specific IgG in vaccinated guinea pigs. After the completion of vaccination, but just prior to the aerosol challenge with M. tuberculosis, blood was harvested from each animal to obtain sera. The levels of IgG antibodies reactive to heat-inactivated M. tuberculosis or to recombinant PknD sensor were determined for each group. While sera from BCG-vaccinated animals demonstrated high levels of IgG antibodies reactive to heat-inactivated M. tuberculosis (P<0.01) (panel A), only sera from PknD-vaccinated animals showed high levels of IgG antibodies reactive to PknD (P<0.01) (panel B). Sera from three animals from each group were tested. Each assay was performed in triplicate. Data are presented on a linear scale as mean±standard deviation; and

FIG. 4 shows pre-incubation of M. tuberculosis with sera from PknD-vaccinated guinea pigs reduces invasion of brain microvascular endothelia. Human brain microvascular endothelia are the primary components of the blood-brain barrier and protect the CNS from the systemic circulation. Brain microvascular endothelial invasion of M. tuberculosis pre-incubated with sera from each vaccinated group normalized to the unvaccinated animals (PBS) is shown. While sera from the BCG vaccinated animals was not protective, pre-incubation of M. tuberculosis with sera from PknD-vaccinated guinea pigs significantly reduced (>10 fold) invasion of brain microvascular endothelia (P<0.01). Sera from three animals from each group were tested. Each assay was performed in triplicate. Data are presented on a linear scale as mean±standard deviation.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. PknD Sensor Domain Vaccines

M. tuberculosis PknD is a transmembrane protein with an extracellular surface exposed (sensor) domain (Good et al, 2004) that has been previously characterized as an important virulence factor required for the pathogenesis of central nervous system (CNS) tuberculosis (TB) in animal models (Be et al., 2012; Be et al., 2008). Further, it also has been demonstrated that the M. tuberculosis PknD sensor domain is sufficient to trigger bacillary invasion of human brain microvascular endothelia, which are the primary components of the blood-brain barrier (BBB) protecting the brain. Moreover, invasion of human brain microvascular endothelia could be neutralized by pre-incubation of bacteria with PknD (sensor)-specific antisera (Be et al., 2012). Further, based on gene sequence analyses, pathogenic strains of M. tuberculosis produce the full PknD. A frame shift mutation in the pknD homolog in BCG, however, results in a predicted truncated protein without the C-terminal sensor domain (Peirs et al., 2000).

The coding sequence for the full-length M. tuberculosis PknD protein is known (UniProtKB/Swiss-Prot: O05871.1; SEQ ID No: 1). The coding sequence for the M. tuberculosis PknD sensor protein is amino acid residues 403-664 (SEQ ID NO: 2) of the full-length PknD protein. The structure of the sensor domain of Mycobacterium tuberculosis PknD has been determined (Good et al., 2004). In two crystal forms, the PknD sensor domain forms a rigid, six-bladed β-propeller with a flexible tether to the transmembrane domain. The PknD sensor domain is the most symmetric β-propeller structure described. All residues that vary most among the blade subdomains cluster in the large “cup” motif, analogous to the ligand-binding surface in many β-propeller proteins. These results suggest that PknD binds a multivalent ligand that signals by changing the quaternary structure of the intracellular kinase domain.

The presently disclosed subject matter relates, at least in part, to the discovery that vaccination with recombinant M. tuberculosis PknD sensor domain offers a viable strategy to protect against CNS TB, including TB meningitis, which is equivalent to BCG in a guinea pig model. Moreover, since BCG lacks the PknD sensor, BCG also could be boosted to develop a more effective vaccine against TB meningitis, a devastating disease that disproportionately affects young children.

A. M. Tuberculosis PknD Sensor Polypeptide Vaccines

In one embodiment, the presently disclosed subject matter relates to a vaccine comprising the M. tuberculosis PknD sensor protein (i.e., an amino acid sequence consisting of SEQ ID NO:2), or an immunogenic fragment thereof. However, those of skill in the art will recognize that the precise primary sequence of the M. tuberculosis PknD sensor protein, or immunogenic fragments thereof, need not be retained in order to successfully practice the presently disclosed subject matter. Various changes in the primary amino acid sequence of the M. tuberculosis PknD sensor protein may be tolerated without compromising the effectiveness of the protein as an antigen. For example, conservative (or even non-conservative) amino acid substitutions are acceptable, or deletions or additions (e.g. at the amino and/or carboxyl termini, or within the protein sequence) may be tolerated.

Accordingly, in one embodiment, the presently disclosed subject matter relates to a vaccine comprising an M. tuberculosis PknD sensor polypeptide, or an immunogenic fragment thereof, wherein the M. tuberculosis PknD sensor polypeptide comprises an amino acid sequence that is at least 70% identical, e.g., at least 75%, 80%, 85%, 90%, 91%, 92%, 93%, 94%, 95%, 96%, 97%, 98%, or 99% identical to the amino acid sequence of SEQ ID NO:2. All such variants are intended to be included in the practice of the presently disclosed subject matter, so long as the resulting variant protein retains the ability to elicit a robust immune response in vivo (i.e., is immunogenic) including humoral and/or cell mediated immune responses, and which result in the inhibition or prevention of M. tuberculosis bacterial invasion of brain microvascular endothelia. Such an immunogenic response may prevent or lessen disease symptoms associated with infection, and preferably will prevent the establishment and maintenance of a persistent, latent Mycobacterial infection in the central nervous system of a vaccinated subject.

Those of skill in the art will recognize that an immunogenic fragment of a protein or polypeptide is a region (usually of contiguous primary structure) which elicits an immune response at least about half as robust, and preferably equal to or superior to, the intact protein or polypeptide. Typically, immunogenic fragments or regions of a protein or polypeptide are those which are surface exposed, and may comprise e.g. external loops, turns, etc. or other secondary structures that are accessible to the immune system. Such determinants, when synthesized outside the framework of the intact protein or polypeptide, may naturally retain sufficient native structure to elicit an immune response, or may be designed according to methods that are well known so as to retain sufficient secondary structure to do so.

Polypeptides suitable in the presently disclosed subject matter may be recombinant or naturally derived. In one embodiment, M. tuberculosis PknD sensor polypeptides suitable presently disclosed subject matter are recombinant and obtained by methods known in the art. Illustratively, a nucleotide sequence may be cloned into a plasmid which is transfected into E. coli and expressed. To ease purification procedures the expressed polypeptides from E. coli optionally include a tag sequence. Illustrative examples of tags suitable for use in the presently disclosed subject matter include poly-histidine, CBP, CYD (covalent yet dissociable NorpD peptide), strep-2, FLAG, HPC or heavy chain of protein C peptide tag, or GST and MBP protein fusion tag systems. It is appreciated that other tag systems are similarly operable. In one embodiment recombinant polypeptides are expressed in E. coli and purified using an affinity tag system followed by enzymatic cleavage of the tag by incorporating an enzyme cleavage site in the expressed polypeptide. Methods of tag cleavage are known in the art and any effective method is appreciated to be suitable for use in the presently disclosed subject matter.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters, including default parameters for pairwise alignments.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al. (1990) J. Mol. Biol. 215:403-410, and DNASTAR (DNASTAR, Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

The term “isolated” designates a biological material (nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated”. The term “purified” does not require the material to be present in a form exhibiting absolute purity, exclusive of the presence of other compounds.

B. DNA Vaccines, Vectors, and Cells

In some embodiments of the presently disclosed subject matter, the vaccine comprises a DNA vaccine. Accordingly, in one embodiment the vaccine comprises a polynucleotide sequence encoding the amino acid sequence of the M. tuberculosis PknD sensor protein (SEQ ID NO:2) or an amino acid sequence an M. tuberculosis PknD sensor polypeptide that is at least 70% identical, e.g., at least 75%, 80%, 85%, 90%, 91%, 92%, 93%, 94%, 95%, 96%, 97%, 98%, or 99% identical to the amino acid sequence of SEQ ID NO:2, or immunogenic fragments thereof. In such embodiments, DNA encoding the polypeptides may be administered as a DNA vaccine, in the form of a vector such as a plasmid, a viral vector (e.g. an adenoviral vector), or other vectors that are known in the art. In addition, the gene encoding the M. tuberculosis PknD sensor polypeptides, or immunogenic fragments thereof, may be contained within a host such as a bacterial host, which is administered to an individual in need of vaccination. The gene may reside on an extrachromosomal element in such a host (e.g. a plasmid) or may be incorporated into the genetic material of the host. Suitable hosts include but are not limited to bacteria (e.g. mycobacteria, Escherichia coli, and others that are well known to those of skill in the art), yeast, etc. Such vectors and hosts may be utilized as vaccine components, and may function as the source of the M. tuberculosis PknD sensor polypeptide or immunogenic fragment thereof that elicits an immune response in the vaccinated subject. Alternatively, such vectors and hosts may be used for other purposes, for example, for experimental research.

In addition, those of skill in the art will recognize that the precise nucleic acid sequences that are originally identified in M. tuberculosis need not be utilized. Due to the redundancy of the genetic code, other alternative nucleic acid sequences may be used to encode the M. tuberculosis PknD sensor polypeptides and immunogenic fragments identified herein. In addition, the nucleic acids that encode the M. tuberculosis PknD sensor polypeptides include DNA, RNA, and various hybrids thereof.

As used herein, a “nucleic acid” or “polynucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” refers to a nucleotide sequence of reduced length relative to the reference nucleic acid and comprising, over the common portion, a nucleotide sequence identical to the reference nucleic acid. Such a nucleic acid fragment according to the presently disclosed subject matter may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000 or 1500 consecutive nucleotides of a nucleic acid according to the presently disclosed subject matter.

As described above, polynucleotides may be used to express the M. tuberculosis PknD sensor polypeptides, e.g., by in vitro transcription, polymerase chain reaction amplification, or cellular expression. The polynucleotide may be DNA and/or RNA and may be single-stranded or double-stranded. In one embodiment, the polynucleotide is a vector which may be used to express the desired polypeptide. The vector may be, e.g., a plasmid vector or a viral vector and may be suited for use in any type of cell, such as mammalian, insect, plant, fungal, or bacterial cells. The vector may comprise one or more regulatory elements necessary for expressing the desired polypeptides, e.g., a promoter, enhancer, transcription control elements, etc. Another embodiment of the presently disclosed subject matter relates to a cell comprising a polynucleotide encoding the M. tuberculosis PknD sensor polypeptides of the invention. In another embodiment, the invention relates to a cell comprising the M. tuberculosis PknD sensor polypeptides of the presently disclosed subject matter. The cell may be any type of cell, e.g., mammalian, insect, plant, fungal, or bacterial cells.

Several methods known in the art may be used to propagate a polynucleotide according to the presently disclosed subject matter. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include but are not limited to retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

Vectors may be introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter (see, e.g., Wu et al. (1992) J. Biol. Chem. 267:963; Wu et al. (1988) J. Biol. Chem. 263:14621).

A polynucleotide according to the presently disclosed subject matter can also be introduced in vivo by lipofection. For the past decade, there has been increasing use of liposomes for encapsulation and transfection of nucleic acids in vitro. Synthetic cationic lipids designed to limit the difficulties and dangers encountered with liposome-mediated transfection can be used to prepare liposomes for in vivo transfection of a gene encoding a marker (Feigner et al. (1988) Proc. Natl. Acad. Sci. USA 84:7413; Mackey et al. (1988) Proc. Natl. Acad. Sci. U.S.A. 85:8027; and Ulmer et al. (1993) Science 259:1745). The use of cationic lipids may promote encapsulation of negatively charged nucleic acids, and also promote fusion with negatively charged cell membranes (Feigner et al. (1989) Science 337:387). Particularly useful lipid compounds and compositions for transfer of nucleic acids are described in PCT Patent Pubs. WO95/18863 and WO96/17823, and in U.S. Pat. No. 5,459,127. The use of lipofection to introduce exogenous genes into the specific organs in vivo has certain practical advantages. Molecular targeting of liposomes to specific cells represents one area of benefit. It is clear that directing transfection to particular cell types would be particularly preferred in a tissue with cellular heterogeneity, such as pancreas, liver, kidney, and the brain. Lipids may be chemically coupled to other molecules for the purpose of targeting (Mackey et al. (1988) Proc. Natl. Acad. Sci. U.S.A. 85:8027). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Pub. WO95/21931), peptides derived from DNA binding proteins (e.g., PCT Patent Pub. WO96/25508), or a cationic polymer (e.g., PCT Patent Pub. WO95/21931).

It is also possible to introduce a vector in vivo as a naked DNA plasmid (see U.S. Pat. Nos. 5,693,622, 5,589,466 and 5,580,859). Receptor-mediated DNA delivery approaches can also be used (Curiel et al. (1992) Hum. Gene Ther. 3:147; Wu et al. (1987) J. BioL Chem. 262:4429).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the presently disclosed subject matter, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

Enhancers that may be used in embodiments of the presently disclosed subject matter include but are not limited to: an SV40 enhancer, a cytomegalovirus (CMV) enhancer, an elongation factor I (EF 1) enhancer, yeast enhancers, viral gene enhancers, and the like.

Termination control regions, i.e., terminator or polyadenylation sequences, may also be derived from various genes native to the preferred hosts. In one embodiment of the presently disclosed subject matter, the termination control region may comprise or be derived from a synthetic sequence, synthetic polyadenylation signal, an SV40 late polyadenylation signal, an SV40 polyadenylation signal, a bovine growth hormone (BGH) polyadenylation signal, viral terminator sequences, or the like.

The terms “3′ non-coding sequences” or “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “regulatory region” means a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell.

### II. Vaccination Methods

A. Methods for Treating or Preventing CNS TB

In one embodiment, the presently disclosed subject matter relates to a method of treating or preventing CNS TB in a subject in need thereof, the method comprising administering to the subject a therapeutically effective amount of a vaccine comprising an M. tuberculosis PknD sensor polypeptide, or an immunogenic fragment thereof. In a particular embodiment, M. tuberculosis PknD sensor polypeptide, or an immunogenic fragment thereof inhibits or prevents M. tuberculosis bacterial invasion of brain microvascular endothelia.

As used herein, “central nervous system (CNS) tuberculosis (TB)” includes meningitis and intra-cranial tuberculomas, and more generally may refer to the establishment and/or maintenance of persistent, latent infection by Mycobacteriae. With respect to the meaning of “persistent” in this context, Mycobacterium tuberculosis possesses the ability to remain quiescent for long periods, “reactivating” months to decades later to produce clinical disease. Various terms, including persistence, latency, dormancy, non-replicating persistence (NRP), etc. have been used to describe this quiescent physiological state of M. tuberculosis. Latency generally refers to the clinical infection and not the bacteria themselves. It is estimated that one third of the world population are latently infected with M. tuberculosis. Clinically, this stage of infection is silent and is only detected by the use of tuberculin skin tests or ex vivo analogs such as QuantiFERON-TB, etc. Latency can be viewed as an equilibrium that exists between the host and organism. Latent tuberculosis develops after an individual has been exposed to M. tuberculosis, the infection has been established, and an immune response develops to controls the pathogen resulting in a quiescent state. Though it is unclear whether M. tuberculosis can develop a state of true metabolic inactivity, there is great amount of pathologic, epidemiologic and clinical data that demonstrates conclusively that latency does occur clinically. In brief therefore, persistence can be defined as the physiological state in which M. tuberculosis can survive in tissues for months to decades without apparent replication, yet possessing the ability to resume growth and activate months to decades later to cause disease. Furthermore, in some embodiments of the presently disclosed subject matter, the infection that is treated is an infection caused by Mycobacterium tuberculosis. However, those of skill in the art will recognize that the practice of the presently disclosed subject matter need not be limited to the vaccination of infections caused by this species of mycobacteria, but also in vaccinations by other mycobacteria and Actinomycetes as well.

Any of the M. tuberculosis PknD sensor polypeptides, or immunogenic fragments thereof as described herein may be used within these methods of treating CNS TB in a subject in need thereof. In one embodiment, the vaccine comprises the M. tuberculosis PknD sensor protein (i.e., an amino acid sequence consisting of SEQ ID NO:2), or an immunogenic fragment thereof. In another embodiment, the vaccine comprises an M. tuberculosis PknD sensor polypeptide comprises an amino acid sequence that is at least 70% identical, e.g., at least 75%, 80%, 85%, 90%, 91%, 92%, 93%, 94%, 95%, 96%, 97%, 98%, or 99% identical to the amino acid sequence of SEQ ID NO:2, or an immunogenic fragment thereof.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% inhibition of M. tuberculosis bacterial invasion of brain microvascular endothelia in an assay.

In any of the above-described methods, the administering of any of the disclosed vaccines comprising an M. tuberculosis PknD sensor polypeptide, or an immunogenic fragment thereof can result in at least about a 10%, 15%, 20%, 25%, 30%, 35%, 40%, 45%, 50%, 55%, 60%, 65%, 70%, 75%, 80%, 85%, 90%, 95%, or even 100% decrease in one or more (e.g., 1, 2, 3, 4, 5, 6, 7, 8, 9, or 10) symptoms of CNS TB, compared to a subject that is not administered the disclosed vaccines comprising an M. tuberculosis PknD sensor polypeptide, or an immunogenic fragment thereof.

In any of the above-described methods, the administering of any of the disclosed vaccines comprising an M. tuberculosis PknD sensor polypeptide, or an immunogenic fragment thereof can result in at least about a 10%, 15%, 20%, 25%, 30%, 35%, 40%, 45%, 50%, 55%, 60%, 65%, 70%, 75%, 80%, 85%, 90%, 95%, or even 100% decrease in the likelihood of developing CNS TB, compared to a control population of subjects that are not administered the disclosed vaccines comprising an M. tuberculosis PknD sensor polypeptide, or an immunogenic fragment thereof.

As used herein, the term “inhibit” or “inhibits” means to decrease, suppress, attenuate, diminish, arrest, or stabilize the development or progression of a disease, disorder, or condition, the activity of a biological pathway, or a biological activity such as bacterial invasion of brain microvascular endothelia, e.g., by at least 10%, 20%, 30%, 40%, 50%, 60%, 70%, 80%, 90%, 95%, 98%, 99%, or even 100% compared to an untreated control subject, cell, biological pathway, or biological activity. By the term “decrease” is meant to inhibit, suppress, attenuate, diminish, arrest, or stabilize a symptom of a CNS TB disease, disorder, or condition. CNS TB disease associated symptoms include, but are not limited to pulmonary symptoms and/or neurologic deficits, signs and symptoms of increased intracranial pressure or space-occupying lesions in the brain or spine such as headache, stiff neck, fever, weight loss, blurry vision, confusion, lethargy, nausea, vomiting, and, for spinal cord lesions, lower extremity weakness or bowel or bladder symptoms. Symptoms of meningitis may include altered mental status, fever, seizure, cranial nerve deficits, papilledema, or meningismus. Symptoms of tuberculomas may include cranial nerve deficits, altered mental status, visual changes, hemiparesis, or seizures, while subjects with spinal cord lesions will have a physical examination consistent with the location of the lesion in the spinal cord. It will be appreciated that, although not precluded, treating a disease, disorder or condition does not require that the disease, disorder, condition or symptoms associated therewith be completely eliminated.

The method described above for treating or preventing sickle cell disease in a subject in need thereof may be carried out using a single aptamer targeted to HbS, or may be carried out using two or more different aptamers targeted to HbS, e.g., three, four, five, or six different aptamers.

For use within the methods for treating or preventing CNS TB in a subject in need thereof, the vaccines comprising M. tuberculosis PknD sensor polypeptides described herein optionally be administered in conjunction with other compounds (e.g., therapeutic agents such as other vaccines) or treatments useful in treating CNS TB. The other compounds or treatments may optionally be administered concurrently. As used herein, the word “concurrently” means sufficiently close in time to produce a combined effect (that is, concurrently may be simultaneously, or it may be two or more events occurring within a short time period before or after each other). The other compounds may be administered separately from the vaccines comprising M. tuberculosis PknD sensor polypeptides as disclosed herein, or may be combined together with the vaccines comprising M. tuberculosis PknD sensor polypeptides as disclosed herein in a single composition.

In one embodiment, the method of treating or preventing CNS TB in a subject in need thereof comprises administering to the subject a therapeutically effective amount of a vaccine comprising an M. tuberculosis PknD sensor polypeptide, or an immunogenic fragment thereof, further comprising administering an additional vaccine to the subject, particularly wherein the additional vaccine is BCG. In one embodiment, administration of the vaccine comprising an M. tuberculosis PknD sensor polypeptide, or an immunogenic fragment thereof is prior to administering BCG. In another embodiment, administration of the vaccine comprising an M. tuberculosis PknD sensor polypeptide, or an immunogenic fragment thereof is subsequent to administering BCG. In another embodiment, administration of the vaccine comprising an M. tuberculosis PknD sensor polypeptide, or an immunogenic fragment thereof is simultaneous with administering BCG.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. Accordingly, as used herein, “treating” means either slowing, stopping or reversing the progression of CNS TB infection, including reducing or eliminating bacterial invasion of brain microvascular endothelia. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, the vaccine comprising an M. tuberculosis PknD sensor polypeptide, or an immunogenic fragment thereof as described herein can be administered prophylactically to prevent the onset of a disease, disorder, or condition, or to prevent the recurrence of a disease, disorder, or condition.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

B. Vaccine Compositions

The presently disclosed vaccine compositions and formulations include vaccine compositions comprising an M. tuberculosis PknD sensor polypeptide, or an immunogenic fragment thereof as described herein, alone or in combination with one or more additional therapeutic agents (such as other vaccines), in admixture with a physiologically compatible carrier, which can be administered to a subject, for example, a human subject, for therapeutic or prophylactic treatment. As used herein, “physiologically compatible carrier” refers to a physiologically acceptable diluent including, but not limited to water, phosphate buffered saline, or saline, and, in some embodiments, include an adjuvant.

Acceptable carriers, excipients, or stabilizers are nontoxic to recipients at the dosages and concentrations employed, and can include buffers such as phosphate, citrate, and other organic acids; antioxidants including ascorbic acid, BHA, and BHT; low molecular weight (less than about 10 residues) polypeptides; proteins, such as serum albumin, gelatin or immunoglobulins; hydrophilic polymers, such as polyvinylpyrrolidone, amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or nonionic surfactants such as Tween, Pluronics, or PEG.

In another embodiment, the vaccine of the presently disclosed subject matter further comprises an adjuvant. In one embodiment, the adjuvant is dimethyl dioctadecyl-ammonium bromide (DDA). Additional adjuvants may include, but are not limited to, monophosphoryl lipid A (MPL); LTK63, lipophilic quaternary ammonium salt-DDA, Trehalose dimycolate and synthetic derivatives, DDA-MPL, DDA-TDM, DDA-TDB, IC-31, aluminum salts, aluminum hydroxyide, aluminum phosphate, potassium aluminum phosphate, Montanide ISA-51, ISA-720, microparticles, immuno stimulatory complexes, liposomes, virosomes, virus-like particles, CpG oligonucleotides, cholera toxin, heat-labile toxin from E. coli, lipoproteins, dendritic cells, IL-12, GM-CSF, nanoparticles illustratively including calcium phosphate nanoparticles, a combination of soybean oil, emulsifying agents, and ethanol to form a nanoemulsion; AS04, ZADAXIN, or combinations thereof.

Compositions to be used for in vivo administration must be sterile, which can be achieved by filtration through sterile filtration membranes, prior to or following lyophilization and reconstitution. Therapeutic compositions may be placed into a container having a sterile access port, for example, an intravenous solution bag or vial having a stopper pierceable by a hypodermic injection needle.

As described above, in certain embodiments, the presently disclosed subject matter also includes combination therapies. Additional therapeutic agents such as additional vaccines, which are normally administered to treat or prevent CNS TB, may be administered in combination with vaccine compositions comprising an M. tuberculosis PknD sensor polypeptide, or an immunogenic fragment thereof as described herein. These additional agents may be administered separately, as part of a multiple dosage regimen, or may be part of a single dosage form, mixed together with the vaccine compositions comprising an M. tuberculosis PknD sensor polypeptide, or an immunogenic fragment thereof as described herein, in a single composition.

By “in combination with” is meant the administration of vaccine compositions comprising an M. tuberculosis PknD sensor polypeptide, or an immunogenic fragment thereof as described herein, with one or more therapeutic agents either simultaneously, sequentially, or a combination thereof. Therefore, a subject administered a combination of vaccines and/or therapeutic agents, can receive a vaccine composition comprising an M. tuberculosis PknD sensor polypeptide, or an immunogenic fragment thereof as described herein, and one or more therapeutic agents (such as an additional vaccine, particularly BCG) at the same time (i.e., simultaneously) or at different times (i.e., sequentially, in either order, on the same day or on different days), so long as the effect of the combination of both agents is achieved in the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the vaccine compositions comprising an M. tuberculosis PknD sensor polypeptide, or an immunogenic fragment thereof as described herein, and one or more therapeutic agents are administered simultaneously, they can be administered to the subject as separate pharmaceutical compositions, or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous or separate administration of the subsequent agent(s).

C. Dosage and Mode of Administration

The presently disclosed vaccine compositions comprising an M. tuberculosis PknD sensor polypeptide, or an immunogenic fragment thereof can be administered using a variety of methods known in the art depending on the subject and the particular disease, disorder, or condition being treated. The administering can be carried out by, for example, intravenous infusion; injection by intravenous, intraperitoneal, intracerebral, intramuscular, intraocular, intraarterial or intralesional routes; or topical or ocular application.

More particularly, as described herein, the presently disclosed vaccine compositions comprising an M. tuberculosis PknD sensor polypeptide, or an immunogenic fragment thereof can be administered to a subject for therapy by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The phrases “systemic administration,” “administered systemically,” “peripheral administration” and “administered peripherally” as used herein mean the administration of vaccine compositions comprising an M. tuberculosis PknD sensor polypeptide, or an immunogenic fragment thereof, a compound, drug or other material other than directly into the central nervous system, such that it enters the patient's system and, thus, is subject to metabolism and other like processes, for example, subcutaneous administration.

The phrases “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

The presently disclosed pharmaceutical compositions can be manufactured in a manner known in the art, e.g. by means of conventional mixing, dissolving, granulating, dragee-making, levitating, emulsifying, encapsulating, entrapping or lyophilizing processes.

More particularly, pharmaceutical compositions for oral use can be obtained through combination of vaccine compositions comprising an M. tuberculosis PknD sensor polypeptide, or an immunogenic fragment thereof with a solid excipient, optionally grinding a resulting mixture, and processing the mixture of granules, after adding suitable auxiliaries, if desired, to obtain tablets or dragee cores. Suitable excipients include, but are not limited to, carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl cellulose; and gums including arabic and tragacanth; and proteins, such as gelatin and collagen; and polyvinylpyrrolidone (PVP:povidone). If desired, disintegrating or solubilizing agents, such as cross-linked polyvinyl pyrrolidone, agar, alginic acid, or a salt thereof, such as sodium alginate, also can be added to the compositions.

Dragee cores are provided with suitable coatings, such as concentrated sugar solutions, which also can contain gum arabic, talc, polyvinylpyrrolidone, carbopol gel, polyethylene glycol (PEG), and/or titanium dioxide, lacquer solutions, and suitable organic solvents or solvent mixtures. Dyestuffs or pigments can be added to the tablets or dragee coatings for product identification or to characterize the quantity of vaccine compositions comprising an M. tuberculosis PknD sensor polypeptide, or an immunogenic fragment thereof, e.g., dosage, or different combinations of doses.

Pharmaceutical compositions suitable for oral administration include push-fit capsules made of gelatin, as well as soft, sealed capsules made of gelatin and a coating, e.g., a plasticizer, such as glycerol or sorbitol. The push-fit capsules can contain active ingredients admixed with a filler or binder, such as lactose or starches, lubricants, such as talc or magnesium stearate, and, optionally, stabilizers. In soft capsules, vaccine compositions comprising an M. tuberculosis PknD sensor polypeptide, or an immunogenic fragment thereof can be dissolved or suspended in suitable liquids, such as fatty oils, liquid paraffin, or liquid polyethylene glycols (PEGs), with or without stabilizers. Stabilizers can be added as warranted.

In some embodiments, the presently disclosed pharmaceutical compositions can be administered by rechargeable or biodegradable devices. For example, a variety of slow-release polymeric devices have been developed and tested in vivo for the controlled delivery of drugs, including proteinacious biopharmaceuticals. Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly(2-hydroxyethyl-methacrylate) (Langer et al., J. Biomed. Mater. Res. 15:167, 1981; Langer, Chem. Tech. 12:98, 1982), ethylene vinyl acetate (Langer et al., Id), or poly-D-(−)-3-hydroxybutyric acid (EP 133,988A). Sustained release compositions also include liposomally entrapped vaccine compositions comprising an M. tuberculosis PknD sensor polypeptide, or an immunogenic fragment thereof, which can be prepared by methods known per se (Epstein et al., Proc. Natl. Acad. Sci. U.S.A. 82:3688, 1985; Hwang et al., Proc. Natl. Acad. Sci. U.S.A. 77:4030, 1980; U.S. Pat. Nos. 4,485,045 and 4,544,545; and EP 102,324A). Ordinarily, the liposomes are of the small (about 200-800 Angstroms) unilamelar type in which the lipid content is greater than about 30 mol % cholesterol, the selected proportion being adjusted for the optimal therapy. Such materials can comprise an implant, for example, for sustained release of the presently disclosed vaccine compositions comprising an M. tuberculosis PknD sensor polypeptide, or an immunogenic fragment thereof, which, in some embodiments, can be implanted at a particular, pre-determined target site.

Pharmaceutical compositions for parenteral administration include aqueous solutions of vaccine compositions comprising an M. tuberculosis PknD sensor polypeptide, or an immunogenic fragment thereof. For injection, the presently disclosed pharmaceutical compositions can be formulated in aqueous solutions, for example, in some embodiments, in physiologically compatible buffers, such as Hank's solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of vaccine compositions comprising an M. tuberculosis PknD sensor polypeptide, or an immunogenic fragment thereof or vehicles include fatty oils, such as sesame oil, or synthetic fatty acid esters, such as ethyl oleate or triglycerides, or liposomes. Optionally, the suspension also can contain suitable stabilizers or agents that increase the solubility of the vaccine compositions comprising an M. tuberculosis PknD sensor polypeptide, or an immunogenic fragment thereof to allow for the preparation of highly concentrated solutions.

For nasal or transmucosal administration generally, penetrants appropriate to the particular barrier to be permeated are used in the formulation. Such penetrants are generally known in the art.

For inhalation delivery, the agents of the disclosure also can be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Additional ingredients can be added to compositions for topical administration, as long as such ingredients are pharmaceutically acceptable and not deleterious to the epithelial cells or their function. Further, such additional ingredients should not adversely affect the epithelial penetration efficiency of the composition, and should not cause deterioration in the stability of the composition. For example, fragrances, opacifiers, antioxidants, gelling agents, stabilizers, surfactants, emollients, coloring agents, preservatives, buffering agents, and the like can be present. The pH of the presently disclosed topical composition can be adjusted to a physiologically acceptable range of from about 6.0 to about 9.0 by adding buffering agents thereto such that the composition is physiologically compatible with a subject's skin.

The presently disclosed subject matter also includes the use of vaccine compositions comprising an M. tuberculosis PknD sensor polypeptide, or an immunogenic fragment thereof disclosed herein, in the manufacture of a medicament for CNS TB.

Regardless of the route of administration selected, the presently disclosed vaccine compositions comprising an M. tuberculosis PknD sensor polypeptide, or an immunogenic fragment thereof are formulated into pharmaceutically acceptable dosage forms such as described herein or by other conventional methods known to those of skill in the art.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition (e.g., a disease, condition, or disorder related to CNS TB), or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

Actual dosage levels of the active ingredients in the presently disclosed vaccine compositions can be varied so as to obtain an amount of the active ingredient that is effective to achieve the desired therapeutic response for a particular subject, composition, route of administration, and disease, disorder, or condition without being toxic to the subject. The selected dosage level will depend on a variety of factors including the activity of the particular vaccine composition comprising an M. tuberculosis PknD sensor polypeptide, or an immunogenic fragment thereof employed, the route of administration, the time of administration, the rate of excretion of the particular vaccine being employed, the duration of the treatment, other drugs, vaccines and/or materials used in combination with the particular vaccine employed, the age, sex, weight, condition, general health and prior medical history of the patient being treated, and like factors well known in the medical arts.

A physician having ordinary skill in the art can readily determine and prescribe the effective amount of the vaccine composition required. Accordingly, the dosage range for administration will be adjusted by the physician as necessary.

Generally, doses of vaccine compositions comprising an M. tuberculosis PknD sensor polypeptide, or an immunogenic fragment thereof will range from about 0.0001 to about 1000 mg per kilogram of body weight of the subject. In certain embodiments, the dosage is between about 1 μg/kg and about 500 mg/kg, more preferably between about 0.01 mg/kg and about 50 mg/kg. For example, in certain embodiments, a dose can be about 1, 5, 10, 15, 20, or 40 mg/kg.

D. Kits or Pharmaceutical Systems

The presently disclosed vaccine compositions comprising an M. tuberculosis PknD sensor polypeptide, or an immunogenic fragment thereof can be assembled into kits or pharmaceutical systems for use in treating or preventing CNS TB. In some embodiments, the presently disclosed kits or pharmaceutical systems include vaccine compositions comprising an M. tuberculosis PknD sensor polypeptide, or an immunogenic fragment thereof disclosed herein. In particular embodiments, the vaccine compositions comprising an M. tuberculosis PknD sensor polypeptide, or an immunogenic fragment thereof, are in unit dosage form. In further embodiments, the vaccine compositions comprising an M. tuberculosis PknD sensor polypeptide, or an immunogenic fragment thereof, can be present together with a pharmaceutically acceptable solvent, carrier, excipient, or the like, as described herein.

In some embodiments, the presently disclosed kits comprise one or more containers, including, but not limited to a vial, tube, ampule, bottle and the like, for containing the vaccine composition. The one or more containers also can be carried within a suitable carrier, such as a box, carton, tube or the like. Such containers can be made of plastic, glass, laminated paper, metal foil, or other materials suitable for holding medicaments.

In some embodiments, the container can hold a composition that is by itself or when combined with another composition effective for treating or preventing the condition and may have a sterile access port (for example the container may be an intravenous solution bag or a vial having a stopper pierceable by a hypodermic injection needle). Alternatively, or additionally, the article of manufacture may further include a second (or third) container including a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The presently disclosed kits or pharmaceutical systems also can include associated instructions for using the vaccine compositions comprising an M. tuberculosis PknD sensor polypeptide, or an immunogenic fragment thereof as disclosed herein for treating or CNS TB. In some embodiments, the instructions include one or more of the following: a description of the vaccine compositions comprising an M. tuberculosis PknD sensor polypeptide, or an immunogenic fragment thereof as disclosed herein; a dosage schedule and administration for treating or preventing CNS TB; precautions; warnings; indications; counter-indications; overdosage information; adverse reactions; animal pharmacology; clinical studies; and references. The instructions can be printed directly on a container (when present), as a label applied to the container, as a separate sheet, pamphlet, card, or folder supplied in or with the container.

The compositions of the presently disclosed subject matter may be administered to individuals at risk of being infected by mycobacteria, or to individuals who have been exposed to mycobacteria, or even to individuals known to be infected with mycobacteria. The compositions and methods of the invention may be especially useful for treating individuals who are immunocompromised (e.g. AIDS patients, patients who are receiving chemotherapy, drug addicts, etc.), or for treating persons who are known to be infected with strains of mycobacteria that are known to be drug resistant.

### III. General Terms

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration, and are not to be construed as limiting in any manner to make compounds of the disclosure by other methods.

### Example 1

Since CNS TB and TB meningitis develop due to extrapulmonary hematogenous dissemination of bacilli, the presently disclosed subject matter investigated whether the extracellular (sensor) surface exposed domain of M. tuberculosis PknD is a viable therapeutic target against TB meningitis. Accordingly, the presently disclosed subject matter utilized a guinea pig model with reliable bacillary dissemination to the brain following aerosol challenge with M. tuberculosis (Be et al., 2011). This model was used to compare the protective efficacy of recombinant M. tuberculosis PknD sensor protein with that of BCG against M. tuberculosis dissemination to the brain.

The presently disclosed subject matter, in some embodiments, demonstrates that BCG vaccination limited the pulmonary bacillary burden after aerosol challenge with virulent M. tuberculosis in guinea pigs and also reduced bacillary dissemination to the brain (P=0.01). PknD vaccination also offered significant protection against bacterial dissemination to the brain, which was no different from BCG (P>0.24), even though PknD vaccinated animals had almost 100-fold higher pulmonary bacterial burdens. Higher levels of PknD-specific IgG were noted in animals immunized with PknD, but not in BCG-vaccinated or control animals. Further, pre-incubation of M. tuberculosis with sera from PknD-vaccinated animals, but not with sera from BCG-vaccinated or control animals, significantly reduced bacterial invasion in a human blood-brain barrier model (P<0.01).

### Materials and Methods

Ethics Statement:

All animal procedures have been approved by the ethics committee of Johns Hopkins University.

Vaccination:

Recombinant M. tuberculosis PknD sensor protein was expressed as described previously (Be et al., 2012). Briefly, the coding sequence for PknD amino acid residues 403-664 was cloned into pDEST17 (6×N-terminal his-tag) using the Gateway cloning system (Invitrogen, Grand Island, N.Y.). Expression of PknD protein was induced using 0.1% L-arabinose at 37° C. in BL21-AI E. coli. PknD protein was purified by SDS-PAGE. Vaccinations were performed subcutaneously at 10-, 7-, and 4-weeks prior to aerosol challenge. Animals were divided into four groups: negative controls, injected with equal volumes of PBS; positive controls, injected with a single dose of M. bovis BCG (Danish strain 1331) [5×10⁴ colony-forming units (CFU) in a total volume of 200 μL] 10 weeks prior to aerosol challenge; adjuvant group injected with 20 μg of dimethyldioctadecylammonium bromide [DDA (Sigma-Aldrich, St. Louis, Mo.)]; and PknD group injected with 20 μg of recombinant M. tuberculosis PknD sensor protein in combination with 20 μg of DDA. After vaccination, but prior to infection, blood also was harvested to obtain sera.

Animal Infection:

Hartley guinea pigs (200-250 g) (Charles River, Wilmington, Mass.) were aerosol-infected with frozen titrated bacterial stocks of M. tuberculosis CDC1551 (grown to OD₆₀₀ of 1.0), using the Madison chamber (University of Wisconsin, Madison, Wis.) (Ahmad et al., 2011). Three animals were sacrificed one day after infection to determine implantation. Animals were sacrificed at 4- and 6-weeks post-infection to determine bacillary burden. Lungs, and brains were removed aseptically, homogenized and plated on Middlebrook 7H11 agar plates to determine CFU (Be et al., 2011). The entire brains were homogenized into 10 mL of PBS. One mL was diluted into 9 mL (10 fold dilution) and plated separately. The entirety of the remaining 9 mL was plated and used to determine CFU. 2-Thiophenecarboxylic acid hydrazide (TCH) agar (Sigma-Aldrich) selectively allows growth of M. tuberculosis, but inhibits BCG. Therefore, TCH plates were used to determine M. tuberculosis CFU for tissues obtained from BCG vaccinated animals. Four guinea pigs per group were sacrificed at each time-point, except for the DDA group where only three animals were sacrificed at the 6-weeks' time-point. By week 6 post infection, animals displayed greater morbidity than anticipated, and therefore the decision was made to terminate the study at the 6 weeks post-infection time-point. For mortality data, each death represents an animal found dead in the cage, or deemed to be too sick to remain in the study (based on akinesia, labored breathing and malaise) by the veterinary care staff. Organs from animals found dead in the cages were not harvested for CFU.

Cell Proliferation and IFN-γ Assays:

Guinea pig splenocytes were isolated by disaggregation of spleens, viability assayed using trypan blue (Sigma-Aldrich, USA) and cultured in triplicate with either 10⁶ CFU of heat-inactivated M. tuberculosis CDC1551, 10 μg of recombinant PknD sensor protein, or culture media alone (control), at a concentration of 1×10⁶ cells/mL in a total volume of 200 μL. After 72 h of incubation, culture supernatants were sampled for determination of IFN-γ concentrations using an anti-guinea pig IFN-using an immunosorbent kit (Uscn Life Science Inc., Wuhan, China) as described previously (Hiraishi et al., 2011). Cell proliferation was measured using the Cell Proliferation Reagent WST-1 (Roche, Indianapolis, Ind.) per manufacturer's instructions. Briefly, cells grown in a 96 well plate were incubated for 1 hour at 37° C., 5% CO₂ with 20 μL of the WST-1 reagent, before absorbance was measured at 450 nm. Data were normalized to the culture media alone (control). Data are presented as stimulation index, defined as the proliferation observed in test samples divided by that seen in control samples (culture media alone).

Determination of IgG Antibodies:

At each time-point, 10 mL of blood was obtained via cardiac puncture. Blood was allowed to clot overnight at 4° C. and serum isolated by centrifugation (4000 RPM, 20 minutes, 4° C.). Sera were processed in a 96-well ELISA plates (Thermo Scientific). Plates were coated overnight with recombinant M. tuberculosis PknD (1 μg) in carbonate coating buffer (3.03 g Na₂CO₃, 6 g NaHCO₃ in 1 L dH₂0, pH 9.6) sensor protein or 10 μL of heat-inactivated M. tuberculosis CDC1551 (10⁷ CFU/mL), blocked with 1% bovine serum albumin BSA (weight/volume) (Sigma-Aldrich) and washed with 0.1% Tween 20 (Sigma-Aldrich) and incubated with 1000-fold diluted guinea pig sera for 1 hour at 37° C. Following washing, horseradish peroxidase (HRP) conjugated IgG detection antibody (BD, Franklin Lakes, N.J.) was added and incubated for 1 hour at 37° C., washed again with subsequent addition of the HRP substrate 3, 3′,5,5′-tetramethylbenzidine (TMB) (Sigma-Aldrich). The reaction was stopped after 20 minutes using 1 M H₂SO₄ and absorbance measured at 450 nm. Sera from three animals from each group were tested. Each assay was performed in triplicate.

In Vitro Neutralization Assays:

M. tuberculosis CDC1551 were pre-incubated with guinea pig sera (1:1250 dilution) for 60 minutes. Bacteria were subsequently washed in PBS and used for invasion assays with primary human brain microvascular endothelial cells (HBMEC) as described previously (Be et al., 2012). Sera from three animals from each group were tested. Each assay was performed in triplicate.

Statistical Analysis:

Comparisons between groups were performed using a one tail distribution, two sample unequal variance Student's t test in Excel 2007 (Microsoft) except for CFU data where a one tail Mann-Whitney U test (http://elegans.som.vcu.edu/˜1eon/stats/utest.html) and stimulation indices where a One-way ANOVA with Bonferroni's post-hoc test (Prism 5 version 5.01, GraphPad software, San Diego, Calif.) were utilized. Data are presented on a linear scale as mean±standard deviation (SD) except for CFU, where a logarithmic scale (mean±SD) has been used.

### Results

PknD Vaccination Protects Against M. tuberculosis Dissemination to the Brain:

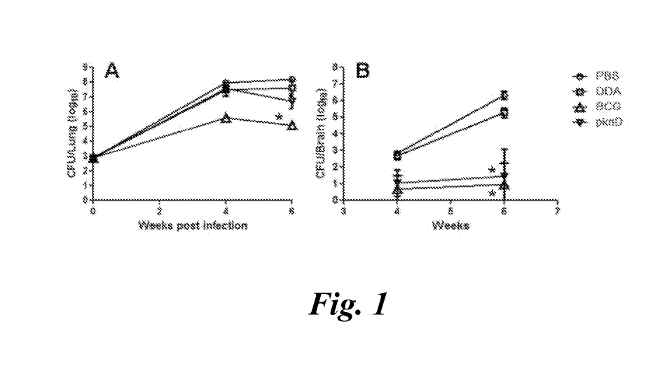

Groups of PknD, BCG vaccinated, unvaccinated (PBS) and animals administered adjuvant only (DDA) were challenged via aerosol with virulent M. tuberculosis and the bacillary burden in the lungs and brains was assessed 4- and 6-weeks after infection. Pulmonary implantation one day after aerosol infection was 2.90±0.06 log₁₀ CFU. Compared with unvaccinated animals (PBS), BCG vaccination significantly limited the pulmonary CFU burden at both 4-weeks (5.61±0.12 versus 7.99±0.12 log₁₀ CFU; P=0.01) and 6-weeks (5.09±0.16 versus 8.20±0.17 log₁₀ CFU; P=0.01) after the aerosol challenge. The pulmonary bacillary burdens in PknD- and DDA-vaccinated groups, however, were similar to the unvaccinated (PBS) group (FIG. 1A).

Whole brains also were harvested from each animal to measure M. tuberculosis dissemination to the CNS. Compared with unvaccinated animals (PBS), BCG vaccination significantly reduced the bacterial burden in the brain at both 4-weeks (0.70±0.40 versus 2.84±0.06 log₁₀ CFU; P=0.01) and 6-weeks (0.97±1.27 versus 6.32±0.25 log₁₀ CFU; P=0.01) after infection. PknD vaccination also significantly reduced the bacterial burden in the brain at both 4-weeks (1.05±0.40 versus 2.84±0.06 log₁₀ CFU; P=0.01) and 6-weeks (1.44±1.66 versus 6.32±0.25 log₁₀ CFU; P=0.01) after infection (FIG. 1B) and this protection was no different than that offered by BCG (P>0.24), even though the PknD vaccinated animals had almost 100-fold higher bacterial burdens in the lungs. These data suggest that vaccination with M. tuberculosis PknD sensor protects against bacterial dissemination to the brain. A trend for lower mortality also was noted in both the BCG (25%; 1 of 4) or PknD (20%; 1 of 5) vaccinated groups compared to both the control groups PBS (71%; 5 of 7) or DDA (100%; 4 of 4), where much higher mortality was noted.

PknD Vaccination Induces Specific IFN-γ and IgG Responses:

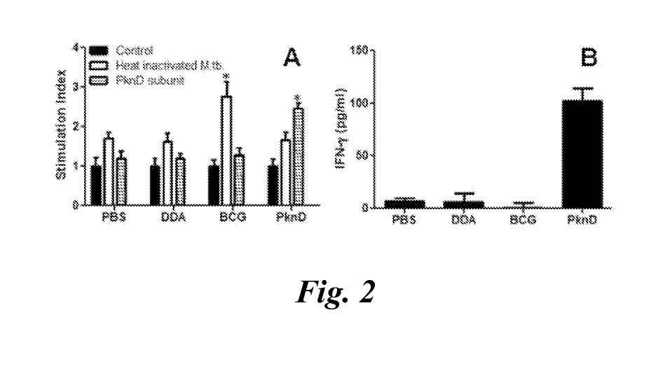

To determine the ability of PknD to induce splenocyte proliferation, cells were isolated from the spleens of animals from each group. Cell preparations were then exposed to culture media (control), heat-inactivated M. tuberculosis or recombinant PknD subunit in the WST-1 assay, which measures formazan formation by the mitochondrial dehydrogenase of viable cells and provides a sensitive and accurate nonradioactive method to measure cell proliferation (Takahashi et al., 1999; Bounous et al., 1992; Cisneros-Lira et al., 2003) (FIG. 2A). Significant cellular proliferation in response to PknD subunit was only observed in splenocytes from PknD vaccinated animals (P=0.002). Similarly, significant proliferation in response to heat-inactivated M. tuberculosis was only observed in splenocytes from BCG vaccinated animals (P=0.01). Supernatants from these stimulation assays were also harvested to determine IFN-γ levels. Consistent with proliferation data, only animals vaccinated with PknD (P<0.01) generated appreciable levels of IFN-γ in response to the PknD subunit protein (FIG. 2B).

PknD Vaccinated Animals have High Levels of PknD-Specific IgG Antibodies:

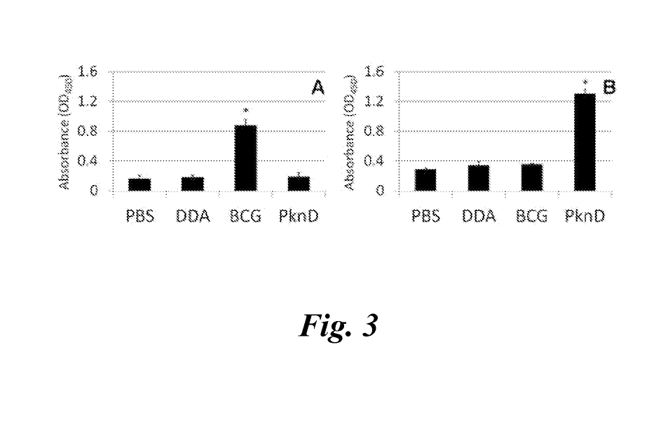

After completion of vaccination, but just prior to aerosol challenge with M. tuberculosis, blood was harvested from each animal to obtain sera. The levels of IgG antibodies reactive to heat-inactivated M. tuberculosis or to recombinant PknD sensor were determined for each group. As expected, only sera from BCG-vaccinated animals had high levels of IgG antibodies reactive to heat-inactivated M. tuberculosis (P<0.01) (FIG. 3A). In contrast, only sera from PknD-vaccinated animals had high levels of IgG antibodies reactive to PknD (P<0.01) (FIG. 3B).

Pre-Incubation of M. tuberculosis with Sera from PknD-Vaccinated Guinea Pigs Reduces Invasion of Brain Microvascular Endothelia:

Human brain microvascular endothelia are the primary components of the blood-brain barrier and protect the central nervous system (Rubin and Staddon, 1999). It has been previously demonstrated that invasion of human brain microvascular endothelia by M. tuberculosis can be neutralized by PknD (sensor)-specific antiserum (Be et al., 2012).

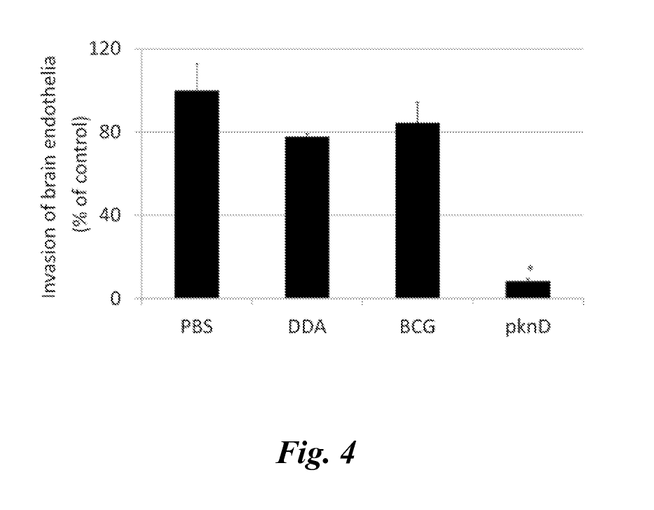

To further assess whether sera from PknD-vaccinated animals were indeed protective, whether pre-incubation of M. tuberculosis with guinea pig sera reduced invasion of the brain microvascular endothelia was investigated. FIG. 4 demonstrates the invasion of M. tuberculosis pre-incubated with sera from each vaccinated group normalized to the unvaccinated animals (PBS). While sera from the BCG-vaccinated animals was not protective, pre-incubation of M. tuberculosis with sera from PknD-vaccinated guinea pigs significantly reduced (>10 fold) invasion of brain microvascular endothelia (P<0.01). Collectively, these data suggest that PknD vaccinated animals produced a robust PknD-specific IgG response that also prevents invasion of brain microvascular endothelia.

### Discussion

The presently disclosed subject matter investigated whether vaccination strategies targeting M. tuberculosis PknD (sensor) can serve as therapeutic methods to protect against CNS TB and TB meningitis. To test this hypothesis, an aerosol challenge guinea pig model with hematogenous bacterial dissemination to the brain (Be et al., 2011) was utilized. Following vaccination, all animal groups were challenged, via aerosol, with virulent M. tuberculosis and the bacterial burden assessed 4- and 6-weeks after infection. Consistent with prior data (WHO, 1993; Ordway et al., 2008), BCG vaccination significantly limited the pulmonary bacterial burden after the aerosol challenge, and also significantly reduced bacterial dissemination to the brain. While vaccination with PknD did not affect the bacterial burden in the lungs at 4-weeks and only modestly decreased bacterial burden in the lungs at 6-weeks, it nonetheless significantly reduced bacterial dissemination to the brain.

Although the modest decrease in pulmonary bacterial burden in the PknD vaccinated group at 6-weeks could have partially contributed to the decreased bacterial dissemination to the CNS, highly significant differences were noted in bacterial dissemination to the CNS even at 4-weeks, when the pulmonary bacterial burden in the PknD vaccinated groups were no different than the controls. In fact, even though the PknD vaccinated animals had an almost 100-fold higher bacterial burden in the lungs, PknD offered protection similar to BCG at either time-point evaluated in this study (P>0.24). Though these data are consistent with a prior observation of the role of M. tuberculosis PknD in the pathogenesis of CNS disease (with no corresponding role in the lung tissues), it should be noted that other extrapulmonary sites such as spleen were not evaluated in the presently disclosed study. Therefore, the possibility of a more generalized extrapulmonary protection offered by PknD vaccination cannot be ruled out. Further, a trend for lower mortality was noted in both the BCG or PknD vaccinated groups. These data were not statistically significant, however, due to the low number of animals in the presently study since determination of mortality rates amongst the different vaccination groups was not one of the end-points.

While previous efforts at developing TB vaccines have focused on the generation of cellular responses; several studies have demonstrated the role of antibodies in protection against pulmonary TB (Glatman-Freedman and Casadevall, 1998; Teitelbaum et al., 1998; Roy et al., 2005), although none have explored the ability to prevent CNS TB. Since TB meningitis develops subsequent to hematogenous dissemination of bacteria (Rich and McCordock, 1933), and the surface exposed PknD sensor is required for invading the BBB that protects the CNS from the systemic circulation (Good et al., 2004; Be et al., 2012; Lopez et al., 2003; Mawuenyega et al., 2005), it was thought that antibody-mediated humoral immunity against PknD could be protecting against brain dissemination in the PknD vaccinated animals. The presently disclosed data indicate that a robust PknD-specific IFN-γ response and higher levels of PknD-specific IgG were noted in animals vaccinated with PknD, but not in those vaccinated with BCG or control animals.

Furthermore, the presently disclosed data also demonstrate that pre-incubation of M. tuberculosis with sera from PknD-vaccinated, but not sera from BCG-vaccinated or control animals could neutralize bacterial invasion of brain microvascular endothelia. Collectively, these data indicate that components of sera, likely PknD-specific IgG, protect against bacillary dissemination to the brain in PknD-vaccinated animals.

One potential limitation of the presently disclosed study is that unpurified sera, as opposed to purified PknD-specific antibodies for the inhibition assays, was utilized. High dilutions of sera, however, were utilized to minimize any non-specific interactions. Further, at this time, the antibody subclass that is potentially involved in neutralizing bacterial invasion of brain microvascular endothelia is not known. Finally, guinea-pigs are outbred, which precluded the possibility of performing passive transfer experiments, which may have further strengthened the presently disclosed data. This limitation may be overcome by utilizing inbred species, such as mice.

Since the major reason for BCG vaccination at birth is for its protective effect against TB meningitis during infancy (WHO, 1993), vaccination with the PknD subunit could offer similar, but more consistent protection against CNS TB, especially for immunosuppressed infants in the setting of HIV. Moreover, since BCG does not express the sensor domain of PknD (Peirs et al., 2000), current BCG vaccines also could be boosted by either developing a recombinant strain that expresses PknD, or by concomitant vaccination with PknD subunit in combination with BCG.

In summary, the presently disclosed subject matter demonstrates that vaccination with recombinant PknD sensor protein protects against bacterial dissemination to the brain in a guinea pig model. Moreover, this protection is likely mediated by humoral IgG responses against PknD (sensor) that prevent the invasion of brain microvascular endothelia. These data are expected to help in developing better vaccines against TB meningitis, a devastating disease that disproportionately affects young children.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Ahmad Z, Fraig M M, Pinn M L, Tyagi S, Nuermberger E L, et al. (2011) Effectiveness of tuberculosis chemotherapy correlates with resistance to Mycobacterium tuberculosis infection in animal models. J Antimicrob Chemother 66: 1560-1566.
Be N, Bishai W, Jain S (2012) Role of Mycobacterium tuberculosis pknD in the Pathogenesis of central nervous system tuberculosis. BMC Microbiology 12: 7.
Be N, Lamichhane G, Grosset J, Tyagi S, Cheng Q, et al. (2008) Murine model to study Invasion and Survival of Mycobacterium tuberculosis in the Central Nervous System. J Infect Dis 198: 1520-1528.
Be N A, Klinkenberg L G, Bishai W R, Karakousis P C, Jain S K (2011) Strain-dependent CNS dissemination in guinea pigs after Mycobacterium tuberculosis aerosol challenge. Tuberculosis (Edinb) 91: 386-389.
Berenguer J, Moreno S, Laguna F, Vicente T, Adrados M, et al. (1992) Tuberculous meningitis in patients infected with the human immunodeficiency virus. N Engl J Med 326: 668-672.
Bounous D I, Campagnoli R P, Brown J (1992) Comparison of MTT colorimetric assay and tritiated thymidine uptake for lymphocyte proliferation assays using chicken splenocytes. Avian Dis 36: 1022-1027.
Campuzano J, Aguilar D, Arriaga K, Leon J C, Salas-Rangel L P, et al. (2007) The PGRS domain of Mycobacterium tuberculosis PE_PGRS Rv1759c antigen is an efficient subunit vaccine to prevent reactivation in a murine model of chronic tuberculosis. Vaccine 25: 3722-3729.
Cisneros-Lira J, Gaxiola M, Ramos C, Selman M, Pardo A (2003) Cigarette smoke exposure potentiates bleomycin-induced lung fibrosis in guinea pigs. Am J Physiol Lung Cell Mol Physiol 285: L949-956.
Girgis N I, Sultan Y, Farid Z, Mansour M M, Erian M W, et al. (1998) Tuberculosis meningitis, Abbassia Fever Hospital-Naval Medical Research Unit No. 3-Cairo, Egypt, from 1976 to 1996. Am J Trop Med Hyg 58: 28-34.
Glatman-Freedman A, Casadevall A (1998) Serum therapy for tuberculosis revisited: reappraisal of the role of antibody-mediated immunity against Mycobacterium tuberculosis. Clin Microbiol Rev 11: 514-532.
Good M C, Greenstein A E, Young T A, Ng H L, Alber T (2004) Sensor domain of the Mycobacterium tuberculosis receptor Ser/Thr protein kinase, PknD, forms a highly symmetric beta propeller. J Mol Biol 339: 459-469.
Hiraishi Y, Nandakumar S, Choi S O, Lee J W, Kim Y C, et al. (2011) Bacillus Calmette-Guerin vaccination using a microneedle patch. Vaccine 29: 2626-2636. 13. Jacobs R F, Starke J R (2003) Mycobacterium tuberculosis. In: Long S S, Pickering L K, Prober C G, editors. Principles and practice of pediatric infectious diseases. 2nd ed. New York: Churchill Livingstone. pp. 796-798.
Jain S K, Kwon P, Moss W J (2005) Management and outcomes of intracranial tuberculomas developing during antituberculous therapy: case report and review. Clin Pediatr (Phila) 44: 443-450.
Katrak S M, Shembalkar P K, Bijwe S R, Bhandarkar L D (2000) The clinical, radiological and pathological profile of tuberculous meningitis in patients with and without human immunodeficiency virus infection. J Neurol Sci 181: 118-126.
Lincoln E M, Sordillo V R, Davies P A (1960) Tuberculous meningitis in children. A review of 167 untreated and 74 treated patients with special reference to early diagnosis. J Pediatr 57: 807-823.
Lopez B, Aguilar D, Orozco H, Burger M, Espitia C, et al. (2003) A marked difference in pathogenesis and immune response induced by different Mycobacterium tuberculosis genotypes. Clin Exp Immunol 133: 30-37.
Mawuenyega K G, Forst C V, Dobos K M, Belisle J T, Chen J, et al. (2005) Mycobacterium tuberculosis functional network analysis by global subcellular protein profiling. Mol Biol Cell 16: 396-404.
Ordway D, Henao-Tamayo M, Shanley C, Smith E E, Palanisamy G, et al. (2008) Influence of Mycobacterium bovis BCG vaccination on cellular immune response of guinea pigs challenged with Mycobacterium tuberculosis. Clin Vaccine Immunol 15: 1248-1258.
Ottenhoff T H, Kaufmann S H (2012) Vaccines against tuberculosis: where are we and where do we need to go? PLoS Pathog 8: e1002607.
Padayatchi N, Bamber S, Dawood H, Bobat R (2006) Multidrug-resistant tuberculous meningitis in children in Durban, South Africa. Pediatr Infect Dis J 25: 147-150.
Padungchan S, Konjanart S, Kasiratta S, Daramas S, ten Dam H G (1986) The effectiveness of BCG vaccination of the newborn against childhood tuberculosis in Bangkok. Bull World Health Organ 64: 247-258.
Parra M, Pickett T, Delogu G, Dheenadhayalan V, Debrie A-S, et al. (2004) The Mycobacterial Heparin-Binding Hemagglutinin Is a Protective Antigen in the Mouse Aerosol Challenge Model of Tuberculosis. Infection and Immunity 72: 6799-6805.
Peirs P, Parmentier B, De Wit L, Content J (2000) The Mycobacterium bovis homologous protein of the Mycobacterium tuberculosis serine/threonine protein kinase Mbk (PknD) is truncated. FEMS Microbiol Lett 188: 135-139.
Rana F S, Hawken M P, Mwachari C, Bhatt S M, Abdullah F, et al. (2000) Autopsy study of HIV-1-positive and HIV-1-negative adult medical patients in Nairobi, Kenya. J Acquir Immune Defic Syndr 24: 23-29.
Rich A R, McCordock H A (1933) The pathogenesis of tuberculous meningitis. Bull Johns Hopkins Hosp 52: 5-37.
Ritz N, Dutta B, Donath S, Casalaz D, Connell T G, et al. (2012) The influence of bacille Calmette-Guerin vaccine strain on the immune response against tuberculosis: a randomized trial. Am J Respir Crit Care Med 185: 213-222.
Romanus V (1987) Tuberculosis in Bacillus Calmette-Guerin-immunized and unimmunized children in Sweden: a ten-year evaluation following the cessation of general Bacillus Calmette-Guerin immunization of the newborn in 1975. Pediatr Infect Dis J 6: 272-280.
Rosenthal S R, Loewinsohn E, Graham M L, Liveright D, Thorne M G, et al. (1961) BCG vaccination in tuberculous households. Am Rev Respir Dis 84: 690-704. 30. Rosenthal S R, Loewinsohne, Graham M L, Liveright D, Thorne G, et al. (1961) BCG vaccination against tuberculosis in Chicago. A twenty-year study statistically analyzed. Pediatrics 28: 622-641.
Roy E, Stavropoulos E, Brennan J, Coade S, Grigorieva E, et al. (2005) Therapeutic efficacy of high-dose intravenous immunoglobulin in Mycobacterium tuberculosis infection in mice. Infect Immun 73: 6101-6109.
Rubin L L, Staddon J M (1999) The cell biology of the blood-brain barrier. Annu Rev Neurosci 22: 11-28.
Sofia M, Maniscalco M, Honore N, Molino A, Mormile M, et al. (2001) Familial outbreak of disseminated multidrug-resistant tuberculosis and meningitis. Int J Tuberc Lung Dis 5: 551-558.
Takahashi K, Orihashi M, Akiba Y (1999) Dietary L-arginine level alters plasma nitric oxide and apha-1 acid glycoprotein concentrations, and splenocyte proliferation in male broiler chickens following Escherichia coli lipopolysaccharide injection. Comp Biochem Physiol C Pharmacol Toxicol Endocrinol 124: 309-314.
Teitelbaum R, Glatman-Freedman A, Chen B, Robbins J B, Unanue E, et al. (1998) A mAb recognizing a surface antigen of Mycobacterium tuberculosis enhances host survival. Proc Natl Acad Sci USA 95: 15688-15693.
Thwaites G E, Hien T T (2005) Tuberculous meningitis: many questions, too few answers. Lancet Neurol 4: 160-170.
Thwaites G E, Nguyen D B, Nguyen H D, Hoang T Q, Do T T, et al. (2004) Dexamethasone for the treatment of tuberculous meningitis in adolescents and adults. N Engl J Med 351: 1741-1751.
Tidjani O, Amedome A, ten Dam H G (1986) The protective effect of BCG vaccination of the newborn against childhood tuberculosis in an African community. Tubercle 67: 269-281.
Udwadia Z F (2012) MDR, XDR, TDR tuberculosis: ominous progression. Thorax 67: 286-288.
Wang B, Henao-Tamayo M, Harton M, Ordway D, Shanley C, et al. (2007) A Toll-like receptor-2-directed fusion protein vaccine against tuberculosis. Clin Vaccine Immunol 14: 902-906.
WHO (1993) The Immunological Basis for Immunization Series: Module 5: Tuberculosis. Geneva: Global Programme for Vaccines and Immunization, World Health Organization.
Young T K, Hershfield E S (1986) A case-control study to evaluate the effectiveness of mass neonatal BCG vaccination among Canadian Indians. Am J Public Health 76: 783-786.

Although the foregoing subject matter has been described in some detail by way of illustration and example for purposes of clarity of understanding, it will be understood by those skilled in the art that certain changes and modifications can be practiced within the scope of the appended claims.

1. A vaccine comprising an M. tuberculosis PknD sensor polypeptide, or an immunogenic fragment thereof, wherein the M. tuberculosis PknD sensor polypeptide comprises an amino acid sequence selected from the group consisting of:
(a) an amino acid sequence at least 80% identical to SEQ ID NO:2;
(b) an amino acid sequence at least 90% identical to SEQ ID NO:2;
(c) an amino acid sequence at least 95% identical to SEQ ID NO:2;
(d) an amino acid sequence at least 99% identical to SEQ ID NO:2; and
(e) an amino acid sequence consisting of SEQ ID NO:2;
wherein the M. tuberculosis PknD sensor polypeptide or immunogenic fragment thereof inhibits or prevents M. tuberculosis bacterial invasion of brain microvascular endothelia.

2. The vaccine of claim 1 wherein said polypeptide is recombinant.

3. The vaccine of claim 1, further comprising an adjuvant.

4. The vaccine of claim 3, wherein the adjuvant is dimethyl dioctadecyl-ammonium bromide (DDA).

5. The vaccine of claim 3, wherein the adjuvant is dimethyl dioctadecyl-ammonium bromide (DDA); monophosphoryl lipid A (MPL); LTK63, lipophilic quaternary ammonium salt-DDA, Trehalose dimycolate and synthetic derivatives, DDA-MPL, DDA-TDM, DDA-TDB, IC-31, aluminum salts, aluminum hydroxyide, aluminum phosphate, potassium aluminum phosphate, Montanide ISA-51, ISA-720, microparticles, immuno stimulatory complexes, liposomes, virosomes, virus-like particles, CpG oligonucleotides, cholera toxin, heat-labile toxin from E. coli, lipoproteins, dendritic cells, IL-12, GM-CSF, nanoparticles illustratively including calcium phosphate nanoparticles, a combination of soybean oil, emulsifying agents, and ethanol to form a nanoemulsion; AS04, ZADAXIN, or combinations thereof.

6. The vaccine of claim 1, wherein the vaccine comprises a physiologically compatible carrier.

7. The vaccine of claim 1, wherein the vaccine further comprises BCG.

8. A method of treating or preventing central nervous system (CNS) tuberculosis (TB) in a subject in need thereof, the method comprising administering to the subject a therapeutically effective amount of a vaccine comprising an M. tuberculosis PknD sensor polypeptide, or an immunogenic fragment thereof, wherein the M. tuberculosis PknD sensor polypeptide comprises an amino acid sequence selected from the group consisting of:
(a) an amino acid sequence at least 80% identical to SEQ ID NO:2;
(b) an amino acid sequence at least 90% identical to SEQ ID NO:2;
(c) an amino acid sequence at least 95% identical to SEQ ID NO:2;
(d) an amino acid sequence at least 99% identical to SEQ ID NO:2; and
(e) an amino acid sequence consisting of SEQ ID NO:2;
wherein the M. tuberculosis PknD sensor polypeptide or immunogenic fragment thereof inhibits or prevents M. tuberculosis bacterial invasion of brain microvascular endothelia.

9. The method of claim 8 wherein said polypeptide is recombinant.

10. The method of claim 8, wherein the vaccine further comprises an adjuvant.

11. The method of claim 10, wherein the adjuvant is dimethyl dioctadecyl-ammonium bromide (DDA).

12. The method of claim 10, wherein the adjuvant is dimethyl dioctadecyl-ammonium bromide (DDA); monophosphoryl lipid A (MPL); LTK63, lipophilic quaternary ammonium salt-DDA, Trehalose dimycolate and synthetic derivatives, DDA-MPL, DDA-TDM, DDA-TDB, IC-31, aluminum salts, aluminum hydroxyide, aluminum phosphate, potassium aluminum phosphate, Montanide ISA-51, ISA-720, microparticles, immuno stimulatory complexes, liposomes, virosomes, virus-like particles, CpG oligonucleotides, cholera toxin, heat-labile toxin from E. coli, lipoproteins, dendritic cells, IL-12, GM-CSF, nanoparticles illustratively including calcium phosphate nanoparticles, a combination of soybean oil, emulsifying agents, and ethanol to form a nanoemulsion; AS04, ZADAXIN, or combinations thereof.

13. The method of claim 8, wherein the vaccine comprises a physiologically compatible carrier.

14. The method of claim 8, further comprising administering an additional vaccine to the subject.

15. The method of claim 14, wherein the additional vaccine is BCG.

16. The method of claim 15, wherein administering the vaccine comprising an M. tuberculosis PknD sensor polypeptide, or an immunogenic fragment thereof is prior to administering BCG.

17. The method of claim 15, wherein administering the vaccine comprising an M. tuberculosis PknD sensor polypeptide, or an immunogenic fragment thereof is subsequent to administering BCG.

18. The method of claim 15, wherein administering the vaccine comprising an M. tuberculosis PknD sensor polypeptide, or an immunogenic fragment thereof is simultaneous with administering BCG.

19. The method of claim 8, wherein the CNS TB is meningitis.

20. The method of claim 8, wherein the CNS TB is intracranial tuberculoma.

21. A kit comprising the vaccine of claim 1, wherein the kit further comprises a set of instructions for administering the vaccine in a therapeutically effective amount for treating or preventing CNS TB in a subject in need thereof.

22. The kit of claim 21, further comprising an additional vaccine, wherein the set of instructions further include methods for concurrent administration.

23. The kit of claim 22, wherein the additional vaccine is BCG.
